(19) United States
(12) Patent Application Publication
(10) Pub. No.: US 2004/0142960 A1
(43) Pub. Date: Jul. 22, 2004

(54) Yohimbine as immunobiologically active agent

(76) Inventors: Enzo Leone, Milano (IT); Georges J.M. Maestroni, Cannobio (VB) (IT)

Correspondence Address: NIXON & VANDERHYE, PC, 1100 N GLEBE ROAD, 8TH FLOOR, ARLINGTON, VA, 22201-4714, US

(21) Appl. No.: 10/732,806

(22) Filed: Dec. 11, 2003

Related U.S. Application Data
(63) Continuation-in-part of application No. PCT/EP02/06317, filed on Jun. 10, 2002.

(30) Foreign Application Priority Data
Jun. 12, 2001 (IT) MI2001A001237

(51) Int. Cl. A61K 31/4745

(52) U.S. Cl. 514/283

(57) ABSTRACT

Use of yohimbine in the preparation of immunobiologically active drugs.

### FIELD OF THE INVENTION

The present invention concerns a method for enhancing the immune response in a subject, which comprises administering to said subject in need of said stimulation an effective dosage of yohimbine, in anyone of its isomeric forms and/or pharmaceutically acceptable acid addition salts or mixtures thereof.

The present invention also relates to the use of yohimbine in the preparation of immunobiologically active drugs, such as vaccines, serums, immunotoxins, antigens, immunoglobulins and immunoglobulin preparations.

### BACKGROUND OF THE INVENTION

The sympathetic neurotransmitter norepinephrine may affect Langerhans cells migration controlled by α1b-adrenergic receptors (G. J M. Maestroni, J. Immunol. 2000, 165:6743). In this publication, the Author discloses that the α₂-adrenergic receptor antagonist yohimbine, administered intraperitoneally, enhances the Langheran's cells migration and that, in general, the migration of dendritic cells to lymph nodes is elicited by yohimbine via endogenous norepinephrine. Nevertheless, in another study, the same Author found that the extent of Th1 differentiation in the response to an antigen can be reduced by the local sympathetic nervous activity in the early phase of dendritic cell stimulation (G. J. M. Maestroni, J. Neuroimmunol. 2002, 129:106). This seemingly controversial findings underline the complexity of the sympathetic nervous system modulation of the immune response.

Langerhans cells express mRNA for the adrenergic receptors α1_{A}, β1 and β2. Epinephrine and norepinephrine could inhibit the ability of Langerhans cells to present antigen for elicitation of contact hypersensitivity response and this effect is inhibited by use of the β2-adrenergic antagonist ICI 118,551 (C., J Seiffert et al. J. Immunol. 2002, 168:6128).

In analogy with Langerhans cells, bone marrow-derived dendritic cells express β1-, β2-, α2_{A}- and α2_{C}-adrenoreceptors genes. The physiological activation of β2-adrenoreceptors in dendritic cells and Langerhans cells results in stimulation of IL-10 which in turn inhibits the chemotactic response to the homeostatic chemokines CCL19 and CCL21 resulting in a reduced antigen-presenting ability (G. J. M. Maestroni et al., J. Neuroimmunol. 2003, 144:91).

Furthermore, it has been reported that CD8+ memory T cells and their product IFN-γ (interferon γ) seem to play a local role in preventing Herpes simplex-1 virus (HSV-1) reactivation (Liu T. et al., Cytokine 2001, Dec 21, 16/6, 205-209). The same observation ad the fact that both CD4+ and CD8+ T cells express adrenergic receptors whose activation increases IFN-γproduction has been disclosed by A. Kohm et al., J. Immunol. 2000, 165, 725-733.

In particular, according to observations of G. J. M. Maestroni, not yet published, the extent of Th1 priming in the adaptive response to a sensitizing allergen seems to depend also on its ability to modulate the local sympathetic nervous activity during the innate immune response and that the major effect of the sympathetic nervous system is to control the innate inflammatory response reducing the ability of antigen presenting cells to prime Th1 cells, that is to induce a cell-mediated immune response. This finding shows that, at the beginning of the immune response, the absence or the lowering of the local sympathetic tone (lowering of epinephrine) has a stimulating action for the cell-mediated Th1 response and is thus coherent with an immunosuppressive action of yohimbine (which conversely increases norepinephrine) in the acute situation of first impact with an antigen, for example with a microbial agent.

### PRIOR ART

Yohimbine (C₂₁H₂₆N₂O₃) is an indolalkylamine alkaloid derived from the bark of Pausinystalia Yohimba, a tree of the Rubiaceae family, of Corynanthe Yohimba, of Pseudocinchona Yohimba, and from the roots of Rauwolfia Serpentina, isolated from the alcoholic extract thereof with other alkaloids having a similar chemical constitution (Chatterjee, Experientia, Vol. X/6 246-247 (1954)). The use of yohimbine as a vasodilator and, at high dosage rates, as a vasoconstrictor, dates back to the last century.

The main yohimbine alkaloids are

yohimbine, (16α,17α)-hydroxyyohimban-16-carboxylic acid methyl ester, m.p. 234-237° C.;

allo-yohimbine, 17-hydroxyyohimban-16β-carboxylic acid methyl ester, m.p. 135-140° C.;

α-yohimbine, 17α-hydroxy-20α-yohimban-16β-carboxylic acid methyl ester, m.p. 243-244° C.

Graham et al., Hypertension, 1982, 4(3), 183-187, disclose the of use yohimbine as an antigenic laboratory tool to prepare anti-yohimbine antibody. In this publication, yohimbine is used as a derivative thereof, covalently bonded to a protein.

Duarte-Moreira et al., Biol. Pharm. Bull. (JP), 2001, 24(2), 201-204, disclose the hydrogen peroxide release in culture of peritoneal macrophage cells of mice treated with a number of natural products and conclude that the results of their test suggest that natural products (mainly tingenone and reserpine and others) with different chemical structure are strong immunological modulators. Among the tested natural products, the Authors cite yohimbine, but the results obtained for the tested compounds make always reference to formulas which do not include yohimbine. In fact, the Authors attributed the name yohimbine to compound No. 15 having a formula which does not correspond to that of yohimbine.

Ckless et al., Automation, 1996, 9(2), 59-65, discloses that yohimbine shows a strong immunosuppressive activity in vitro and proposes this product as an immunosuppressing agent.

Maestroni et al., Non-serial-Mol. Biol. Hematopoiesis, 8th Symposium, 8, 9-13/7/1993, p. 8 and Int. J. Immunopharmacol. 1994, 16(2), 117-122, give the result of a study on the effect of the α1-agrenergic receptor antagonist prazosin on lymphohematopoiesis via noradrenergic modulation. In comparing the concentrations at which the adrenergic antagonists are effective in counteracting norepinephrine, the Authors note that the relative order of potency is prazosin>phentolamine>yohimbine and conclude that the sympathetic innervation of bone marrow cells exerts a general and fine control of hematopoiesis.

Kondo et al., Tohoku J. Exptl. Med., 1999, 189(2), 135-145, disclose the prevention of posttransplant edema in lung allograft by α-adrenergic blockade, for example by pre-treatment with yohimbine. In particular, the document shows that yohimbine improves wet to dry weight ratio (W/D) of weight transplants.

J. S. Jaimes, AIDS Treatment News, Issue No. 159, Sep. 18, 1992, discloses an interview with three persons suffering from AIDS about their successful use of yohimbine to treat severe, long-lasting fatigue and with a physician who prescribed yohimbine to treat fatigue.

Furthermore, the use of yohimbine as an antiviral agent and as an active ingredient of antiviral drugs has already been described, as may be found in EP 1 086 695, incorporated herein by reference.

### SUMMARY OF THE INVENTION

Contrary to literature suggestions based on objective experimental results even obtained by one of the applicants (G. J. M. Maestroni), it has surprisingly been found that yohimbine, in all isomeric forms and/or pharmaceutically acceptable salts or mixtures thereof, has immunizing properties, i.e. in treated subjects it stimulates and enhances the antibody response.

It has also been found that, although yohimbine actually acts as an immunosuppressor in acute situations, for example when administered concurrently with or immediately after an organ transplantation, and could be useful as an adjuvant to the classical immunosuppressive drugs, it has a remarkable immunostimulant activity in chronic situations, namely when the antigen is latent and the subject is asymptomatic.

It has further been found that the immune system may regulate for example HSV-1 reactivation from latency in sensory neurons, and/or limit the transport of virus from sensory ganglia to the peripheral tissues they innervate. In particular, it has been found that a systemic or local treatment with yohimbine results in a local increase of norepinephrine concentration and hence in a stimulation of CD4+ and CD8+ T cells function and IFN-γ production which in turn reduces the HSV-1 reactivation frequency. In addition, the treatment with yohimbine results in a reduced inflammation during HSV-1 reactivation. This finding explains the discovered antiviral activity of yohimbine disclosed in EP 1 086 695, which was very surprising because yohimbine hydrochloride was considered inactive on HSV-1 (B. Alarcón et al., Antiviral Research, 1984, 4, 231-243).

### DETAILED DESCRIPTION OF THE INVENTION

It is an object of the present invention to use yohimbine, in all isomeric forms and/or pharmaceutically acceptable salts or mixtures thereof (hereinbelow referred to as YOHIMBINA), in the preparation of immunobiologically active drugs, such as vaccines, serums, immunotoxins, antigens, and immunoglobulins.

It is a further object of the present invention to use yohimbine, in all isomeric forms and/or pharmaceutically acceptable salts or mixtures thereof, in the preparation of drugs for the stimulation of the immune response, in particular for the prevention and/or treatment of viral diseases, autoimmune diseases, tumours, and allergies.

More particularly, it is an object of the present invention to provide a method for stimulating an enhanced immune response in a subject, which comprises administering to said subject in need of said stimulation an effective dosage of yohimbine, in at least one of its isomeric forms or of a pharmaceutically acceptable acid addition salt thereof or of a solvate thereof.

In this context, the expression “at least” means that yohimbine may be used as yohimbine, allo-yohimbine, α-yohimbine or as a mixture of these forms, as such or as a pharmaceutical acceptable salt or solvate thereof.

Yohimbine exerts an immunizing activity through the stimulation of immune-competent cells, such as for example T lymphocytes, B lymphocytes, dendritic cells.

The dosage is related to the disease intended to be treated with a specific immune-derivative and ranges between 0.1 and 5 mg/kg, to be taken in two or more doses depending on the disease and on the subject.

Also the pre-treatment lasts over a variable period of time (min. eight days), which may be determined by monitoring the dosage of immunoglobulins (Ig) in serum.

Vaccines

The use of YOHIMBINA in the hyperimmunization of donors is at the basis of the new extractive methods of active serums against specific infections, wherefrom antiviral vaccines, specific to each subject and to each disease, will be possibly prepared.

In fact, the treatment with YOHIMBINA enhances the immune response by stimulating the migration of antigen-presenting cells to lymph nodes. Likewise, the response of bone marrow immature elements indicates an increase in their activity.

The so-modified immune memory involves the generation of Ag specific lymphocytic populations with such characteristics as to allow their modification and selection, and, therefore, their use in passive immunization or viruses (also oncogenic) inactivation, and in the production of specific vaccines.

The virus infection is gradually attenuated by repeated passage through YOHIMBINA-pretreated animals, until obtaining—by applying principles analogous to Pasteur's —the so-called fixed virus, whose infection allows the obtainment of vaccine from the animal organs whereto the virus has higher affinity (e.g. the spinal marrow).

Serums

Purified solutions of immunoglobulins present in the serum of subjects immunized against infective diseases (convalescents) are used in passive immunization. Said immunization provides a short term protection and may be homologous or heterologous, depending on the species of the receiver and of the donor.

Therefore, passive immunization produces immediate protection against the dangers of toxins and micro-organisms and is necessary when the generally slow onset of the active antibody response cannot be awaited.

The use of YOHIMBINA in the hyperimmunization of donors is at the basis of the new extractive methods of active serums against specific infections, specific to each subject and to each disease.

In fact, the treatment with YOHIMBINA enhances the immune response through the stimulation of the migration of antigen-presenting dendritic cells to lymph nodes. Likewise, the response of bone marrow immature elements indicates an increase in their activity.

Toxins

The cytotoxicity of antibodies may be increased by conjugating same to cytotoxins; the resultant compounds are denominated immunotoxins. The toxins most widely used are ricin, exotoxin of Pseudomonas and diphtheric toxin. Should said toxins be conjugated to antibodies directed toward target cells, they will become specific and kill only the cells whereto Ab's are directed. The antibodies stimulated by the treatment with YOHIMBINA are qualitatively more efficacious and specific. Furthermore, their selection is less hard from the point of view of the immunogenicity manipulation of Ab and toxic chain. It follows that a decreased hepatotoxicity may be envisaged upon removal of said conjugated compounds.

Antigens

Antigens of basaloma and of epidermis (for transplantation) capable of determining rejection and take, respectively, were selected in vivo and in vitro thanks to cellular cultures of tissues taken from YOHIMBINA-pretreated subjects.

Cellular cultures of tissues taken from YOHIMBINA-pretreated subjects are purified to obtain tissues that can also be grafted into incompatible organisms.

It is a further object of the present invention to use yohimbine, in all isomeric forms and/or pharmaceutically acceptable salts or mixtures thereof, in the preparation of antigens, in particular of antigens for the anti-tumour or anti-rejection treatment. It is a further object of the present invention to use yohimbine, in all isomeric forms and/or pharmaceutically acceptable salts or mixtures thereof, in the cellular culture of tissues for homologous or heterologous transplantation.

Immunoglobulins and Immunoglobulin Preparations

The method is similar to that adopted for the preparation of serums and can be applied to the so-called agammaglobulinaemias, in which normal immunoglobulins (Ig) are deficient or absent, with or without pathological compensation Ig.

In general, due to a genetic defect, lymphoid tissues stop maturing upon the development of epithelial thymus, and the subject is unable to produce immediate or delayed immune response.

The cell-mediated adoptive immunization envisages the transfer of lymphoid cells from a donor immunized, e.g., with a vaccine, to an agammaglobulinemic subject. The donor pretreated with YOHIMBINA acquires an immunological inheritance that qualitatively and quantitatively exceeds that of untreated subjects. With autotransplants or using lymphocytes that do not cause any rejection from the receiver, an immune system, at least partially efficacious, can be reconstituted.

The use of YOHIMBINA in the treatment of viral diseases, such as herpes, was the subject of a clinical trial, which provided experimental evidence on the use of yohimbine in the preparation and commercial scale production of immunobiological drugs.

Said effects are responsible for the enhancement of the immune response during viral infections and tumours, and for the consequent improvement in clinical response.

For example, in the course of a Herpes Simplex Virus (HSV) infection, a reduction in the rate of recurrences takes place, which might be attributed to a higher stabilisation of the antibody response. Therefore, in the presence of a trigger event (physical agents, such as sunlight) or a decreased immune response, there is not a virus migration toward the area of higher sensitivity, with a consequent cutaneous injury, but the persistence of an adequate antibody titre, sufficient to control the replication of same.

The advantages of this method consist in:

the specificity of the antibody response obtained and in its different qualitative characteristics;

the low cost and relatively simple application;

the possibility of using same in proliferative or autoimmune diseases, by selecting the type of the lymphocyte to be cloned.

As mentioned above, in order to stimulate the enhancement of the immune response in a subject in need of said stimulation, YOHIMBINA is administered to said subject in a daily dose of from 0.1 to 5 mg/kg, to be taken in two or more doses, even though a single dose per day can be efficacious in some cases, for example in maintenance therapy. YOHIMBINA may be administered alone or concurrently (separately or in association in the same pharmaceutical preparation) with the specific, classical drugs for the prevention or treatment of each disorder.

In order to provide the expected enhancement of the immune response, YOHIMBINA may be administered in a pharmaceutical composition for oral, parenteral, ophthalmic or topical application, in admixture with a pharmaceutical carrier.

In the pharmaceutical compositions for oral, buccal (or sublingual), subcutaneous, intramuscular, intravenous (or by venous infusion), transdermal, intraocular, topical or rectal administration, YOHIMBINA, preferably yohimbine, α-yohimbine or the hydrochloride of each of them, as an active ingredient, can be administered in unit forms for administration, mixed with conventional pharmaceutical carriers to animals and humans. Appropriate unit forms for administration comprise the oral form such as tablets, soft or hard gelatine capsules, powders or granulates in sachets, and suitably measured oral solutions or suspensions, forms for subcutaneous, intramuscular, intravenous, intranasal or intraocular administration and forms for rectal administration. Unit dosage forms for a topical application consist of appropriate quantities of, for example, cream, ointment, gel or any other topical form, containing the appropriate dose of active ingredient. Intraocular preparations are also administered in drops.

The active ingredient may also be present in form of one of its complexes with a cyclodextrin, for example α-cyclodextrin, β-cyclodextrin, γ-cyclodextrin, 2-hydroxypropyl-β-cyclodextrin or methyl-β-cyclodextrin.

The active ingredient may also be formulated in the form of microcapsules, optionally with one or more carriers or additives.

In each dosage unit, the active ingredient is present in quantities adapted to expected daily dose. The dosage unit is suitably adjusted according to the dosage and the type of administration intended, so that such a dosage unit contains from 0.1 to 10 mg of YOHIMBINA, preferably yohimbine, α-yohimbine or the hydrochloride of each of them. Dosage unit forms of 0.1, 0.5, 1, 2, 2.4, 2.5, 5 or 6 mg of yohimbine hydrochloride are particularly advantageous. In carrying out the method of the invention said dosage units forms are administered once to four times per day.

The pharmaceutical composition may be administered, alone or as an adjuvant to other medicaments specific for each disorder, to a subject suffering from a disease of the immune system, for example an autoimmune disease, such as psoriasis, rheumatoid arthritis, lupus erythematosus; from a tumor; from a viral disease such as Herpes simplex, in particular due to HSV-1, Herpes zooster, herpetic keratitis, influenza; or an allergy. Compositions comprising YOHIMBINA as an active ingredient may also be used for the prevention or the treatment of relapsing manifestations of the above disorders. These pharmaceutical compositions may also be usefully administered to a subject serum positive for HIV (HIV-1 or HIV-2) in order to stimulate an enhancement of the immune response, thus lowering the risk of a manifestation of AIDS.

Compositions for the local treatment of Herpes labialis comprise 0.25 to 5% of YOHIMBINA, in particular yohimbine hydrochloride or α-yohimbine hydrochloride, alone or in association with an antiviral agent such as acyclovir, in admixture with a common carrier for the preparation of creams, with or without a penetration enhancer. The same composition may also be used for the local treatment of skin disorders due to autoimmune diseases.

Compositions useful as adjuvants in the treatment of influenza or of herpetic keratitis preferably consist of tablets or capsules comprising from 2 to 6 mg, preferably 5 mg, of YOHIMBINA, in particular yohimbine hydrochloride or α-yohimbine hydrochloride in admixture with a common carrier for the preparation of tablets for oral administration.

1. Use of yohimbine, in all isomeric forms and/or pharmaceutically acceptable salts or mixtures thereof, in the preparation of immunobiologically active drugs.

2. The use according to claim 1 in the preparation of drugs for the stimulation of the immune response.

3. The use according to claim 1, wherein the immunobiologically active drugs are selected from the group consisting of vaccines, serums, immunotoxins, antigens and immunoglobulins.

4. Use of yohimbine, in all isomeric forms and/or pharmaceutically acceptable salts or mixtures thereof, in the cellular culture of tissues for homologous or eterologous transplantation.

5. A method for stimulating the enhancement of the immune response in a subject, which comprises administering to said subject in need of said stimulation an effective dosage of yohimbine, in anyone of its isomeric forms or as a mixture of said forms, or a pharmaceutically acceptable salt or solvate thereof.

6. The method of claim 5, wherein said enhancement is stimulated in a subject suffering from a disease of the immune system.

7. The method of claim 5, wherein said enhancement is stimulated in a subject suffering from a tumor.

8. The method of claim 5, wherein said enhancement is stimulated in a subject suffering from a viral disease.

9. The method of claim 8, wherein said viral disease is induced by an Herpes virus.

10. The method of claim 9, wherein said Herpes virus is HSV-1.

11. The method of claim 8, wherein said Herpes virus is Herpes zooster.

12. The method of claim 5, wherein said enhancement is stimulated in a subject serum positive for HIV.

13. The method of claim 5, wherein said enhancement is stimulated in a subject suffering from an allergy.
